(19) United States
(12) Patent Application Publication
(10) Pub. No.: US 2016/0128860 A1
(43) Pub. Date: May 12, 2016

(54) PROPHYLACTIC KNEE BRACE

(71) Applicant: LEATT CORPORATION, Santa Clarita, CA (US)

(72) Inventors: Christopher James LEATT, Durbanville (ZA); Cornelis Uys DE JONGH, Durbanville (ZA); Pieter Andre KEEVY, Durbanville (ZA); Jörn Peter STEFFENS, Oldenburg (DE)

(21) Appl. No.: 14/894,237

(22) PCT Filed: May 26, 2014
(86) PCT No.: PCT/IB2014/061716

§ 371 Date: Nov. 25, 2015

(30) Foreign Application Priority Data
May 28, 2013 (GB) 1309515.3

(51) Int. Cl. A61F 5/01

(52) U.S. Cl. CPC A61F 5/0123; A61F 2005/0137; A61F 2005/0165

(57) ABSTRACT

A knee brace (10) according to the invention includes an upper leg formation (12), a lower leg formation (14) and a hinge mechanism (16) that is disposed entirely on the lateral side of the knee. The knee brace (10) has pressure formations (20, 24,62, 28) pressing on three pressure points: on the lateral aspect of the wearer's upper leg, on the lateral aspect of the wearer's lower leg, and against the knee complex on the medial aspect of the distal extremity of the femur. The three pressure formations are connected to one another by a stiff structure (18, 16) which includes the hinge mechanism (16).

### FIELD OF THE INVENTION

This invention relates to knee braces for inhibiting the risk of injuries to the knee, particularly, but not exclusively, in motorcycling, skiing and other activities involving similar mechanics.

### BACKGROUND TO THE INVENTION

Of the most common causes of knee injuries include: valgus stress/deformation, i.e. outward angulation of the lower leg and foot, which results in medial collateral ligament (MCL) damage and/or damages to meniscuses on lateral side or tibial plateau; and overextension, which results in damage to the anterior cruciate ligament (ACL).

A number of knee braces have been developed to inhibit knee movement of injured knees to restrict knee movement to flexion and extension about a virtual transverse axis and to encourage recovery. These conventional knee braces typically include an upper part that is tightly strapped onto the upper leg well above the knee, a lower part that is tightly strapped onto the lower leg well below the knee, and two hinges that are positioned laterally and medially of the knee on the virtual transverse axis. These knee braces are substantially symmetrical and are configured to avoid applying significant forces on the knee. Some of these knee braces have been adapted to be worn during activities with higher risk of knee injuries, such as off-road motorcycling.

Conventional knee braces have been designed primarily to inhibit knee extension, but the natural flexing movement of the knee is more complex than simple pivotal movement and during natural flexion, the knee does not remain aligned with the transverse axis about which the brace's hinges pivot. One example of the complexity of knee movement beyond simple pivotal movement, is that the tibeal plateau tends to rotate inwards on the femur, during knee extension. This conflict between natural knee flexion and the pivotal movement of conventional knee brace hinges cause discomfort, resistance to knee flexion and wear of the knee braces' hinges.

Some of the other shortfalls experienced when wearing conventional knee braces include: bulk that causes interference with equipment (e.g. that prevents them from being worn inside boots), inadequate engagement with the wearers' thighs and inadequate adjustment to fit different knee and leg shapes.

The present invention seeks to reduce the risk of knee injury, to address the shortfalls of conventional knee braces mentioned above and/or to:
control hyperflexion and hyperextension of the knee and ameliorate overextension;
provide stiffness to protect the knee against side impact and bending of the knee and ameliorate valgus deformation;
allow hinging that does not conflict with natural knee movement;
achieve a stable fit on the wearer's thigh;
achieve a comfortable and effective fit on different knee and leg shapes; and
prolong hinge lifespan.

### SUMMARY OF THE INVENTION

According to the present invention there is provided a protective device which includes:
an upper leg formation that is attachable to the upper leg of a wearer and that includes an upper lateral pressure formation that presses against the lateral aspect of the wearer's upper leg, when the device is worn;
a lower leg formation that is attachable to the lower leg of the same leg of the wearer and that includes a lower lateral pressure formation that presses against the lateral aspect of the wearer's lower leg, when the device is worn; and
a hinge mechanism that is connected to the upper leg formation and to the lower leg formation, said hinge mechanism being configured to allow pivotal movement between the upper leg formation and the lower leg formation about at least one pivot axis that extends generally transverse to the wearer's knee;
wherein said hinge mechanism is disposed entirely on the lateral side of the wearer's knee, when the device is worn; said upper leg formation includes a medial pressure formation that presses against the knee complex at the medial aspect of the distal extremity of the femur, when the device is worn; and said upper lateral pressure formation, medial pressure formation and lower lateral pressure formation are connected to one another by a stiff structure which includes the hinge mechanism.

The hinge mechanism may comprise a link that is pivotally connected to the upper leg formation to pivot relative to the upper leg formation about an upper pivot axis, said link also being pivotally connected to the lower leg formation to pivot relative to the lower leg formation about a lower pivot axis, said upper and lower pivot axes extending generally parallel to each other and extending generally transverse to the wearer's knee.

The hinge mechanism may include an upper rotational element that is fixed relative to the upper leg formation and a lower rotational element that is fixed relative to the lower leg formation and the upper and lower rotational elements may be connected together for counter-rotation. The hinge mechanism may include a flexible tensile element that extends partially around the upper and lower rotational elements and crosses between the upper and lower rotational elements, in a figure-of-eight configuration.

The lower leg formation may comprise a thin plate of stiff material.

The upper leg formation may include elongate tensile elements that extend across the posterior aspect of the wearer's thigh in a crossed configuration and the lower leg formation may include elongate tensile elements that extend across the posterior and lateral aspects of the wearer's calf in a crossed configuration, when the device is worn.

### BRIEF DESCRIPTION OF THE DRAWINGS

For a better understanding of the present invention, and to show how it may be carried into effect, the invention will now be described by way of non-limiting example, with reference to the accompanying drawings in which:

FIG. 1 is an anterior view of a first embodiment of a protective device for a left leg of a wearer, in accordance with the present invention;

FIG. 2 is an anterior view of a stiff pivotal structure of the protective device of FIG. 1;

FIG. 3 is an oblique lateral-anterior view of the protective device of FIG. 1, with a sock omitted and with a detail sectional view of a hinge mechanism of the protective device;

FIG. 4 is a lateral view of the pivotal structure of FIG. 2, in flexed and extended conditions;

FIG. 5 is a medial view of a second embodiment of a protective device for a left leg of a wearer, in accordance with the present invention (with straps omitted);

FIG. 6 is an anterior view of the protective device of FIG. 5; and

FIG. 7 is lateral view of the protective device of FIG. 5.

### DETAILED DESCRIPTION OF THE DRAWINGS

Referring to the drawings, a protective device in the form of a knee brace in accordance with the present invention is generally identified by reference numeral 10. Features that are common between the two illustrated embodiments of the invention are identified by the same reference numerals and suffixes are used to distinguish between different embodiments, where necessary.

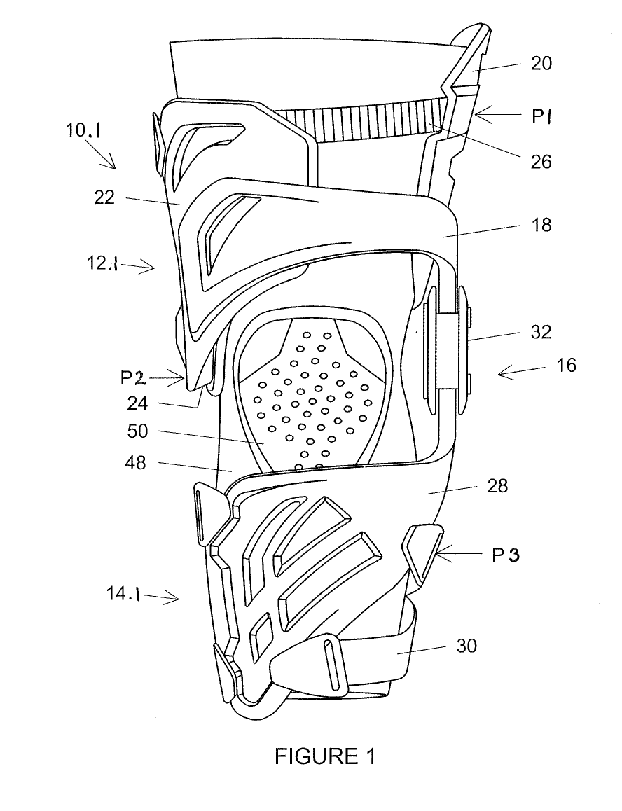

Referring to FIGS. 1 to 4, the knee brace 10.1.1 includes an upper leg formation 12 that is configured to be attached to a wearer's thigh, a lower leg formation 14 that is configured to be attached to the wearer's calf, and a single, lateral hinge mechanism 16 through which the upper and lower leg formations are pivotally connected.

The upper leg formation 12 includes a frame, which in the illustrated embodiment is a curved frame 18 of a suitably stiff (preferably rigid) material, which extends from the hinge mechanism 16 on the lateral aspect of the knee, over the anterior of the thigh. The upper leg formation 12 further includes an upper lateral pressure formation in the form of a lateral thigh pad 20 that extends along the lateral aspect of the wearer's thigh and presses against the thigh, as well as a medial thigh pad 22 that extends along the medial aspect of the thigh, but that is positioned lower than the lateral thigh pad 20. Each of the thigh pads 20,22 is attached to the frame 18 in a manner that allows forces exerted by the frame to be transferred directly to the thigh pads and to be exerted by the thigh pads as pressure on the wearer's thigh.

The lower end of the medial thigh pad 22 can serve as a medial pressure formation that presses against the medial aspect of the upper leg in the region of the knee, e.g. on the medial aspect of the distal extremity of the femur. However, in the illustrated embodiment, the medial thigh pad 22 includes a medial pressure formation in the form of a protuberance or knee pad 24 that is adjustable to compensate for variations in wearers' knee anatomies and to move the medial pressure formation into contact with the knee complex at the medial aspect of the distal extremity of the femur—referred to herein as the medial pressure point.

The lateral thigh pad 20 and medial thigh pad 22 (with the knee pad 24) are attached to the frame 18 and are held in position on the wearer's thigh, e.g. with elongate tensile elements in the form or straps 26 that extend across the posterior of the thigh in a crossed or other (e.g. circumferential) configuration. The large thigh pads 20,22 distribute pressure over a large area and the crossed configuration of the straps 26 allows the upper leg formation 12 to be attached to the upper leg comfortably, but firmly, while allowing normal muscle action, without undue pressure on the muscles.

The lower leg formation 14 includes a plate 28 of a suitably stiff (preferably rigid) material, which extends from the hinge mechanism 16, along the lateral aspect of the knee and calf, over the anterior of the shin. The plate 28 is wider and quite thin in the shin region and is attached to the wearer's calf by elongate tensile elements in the form or straps 30 that extend across the posterior and lateral aspects of the calf in a crossed or other configuration—which prevents undue pressure on the calf muscles. The thin profile of the plate 28 and straps 30 allow the lower leg formation to be worn with boots. (The “plate” 28 is reasonably thick in the vicinity of the hinge mechanism 16 and does not truly resemble a “plate” in this region, but the name “plate” is used herein to refer to the entire part—for simplicity.)

The straps 26,30 can be tightened with ratchet mechanisms (not shown in the drawings) and can be adjusted easily to ensure that the knee brace 10.1 fits comfortably and tightly on various leg sizes.

The plate 28 forms a lateral pressure formation below its attachment to the hinge mechanism 16, where it presses against the lateral aspect of the calf. The knee brace 10.1 thus includes at least three points where it can exert pressure on the wearer's leg: with the lateral thigh pad 20, knee pad 24 and lateral pressure formation of the plate 28. These three points are shown with arrows on FIG. 1, marked “P” (with suffixes), and by exerting pressure on the leg at the three pressure points, the knee brace 10.1 stabilises the knee against valgus stress and thus against meniscus and MCL injury. The pressure points P include a proximal lateral pressure point P1, laterally on the thigh; the medial pressure point P2; and a distal lateral pressure point P3, laterally on the calf.

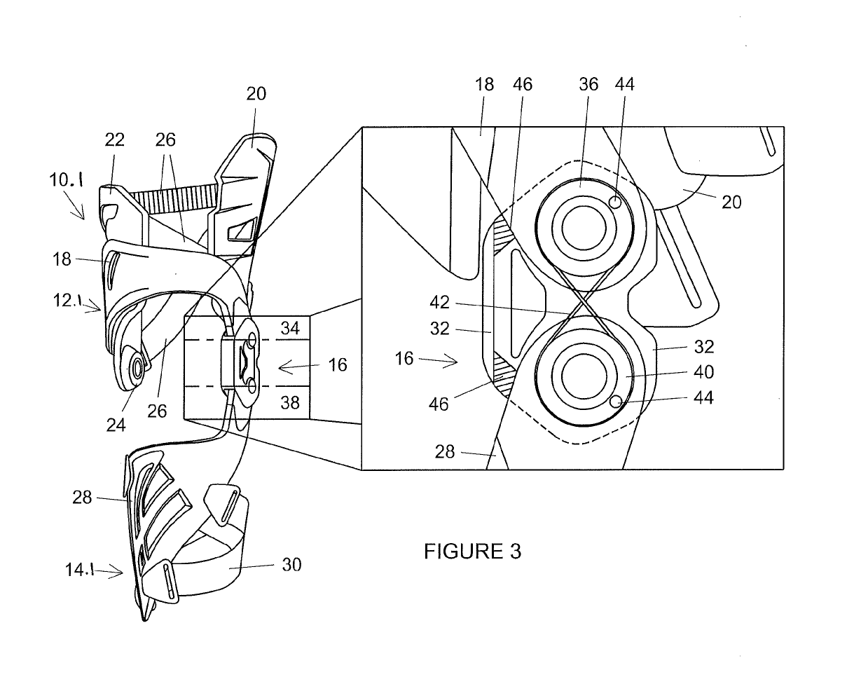

The hinge mechanism 16 includes a housing 32 that forms a link between the upper and lower leg formations 12,14. As shown in FIG. 3, a lower end of the frame 18 of the upper leg formation 12 extends inside the housing 32 and is pivotally connected to the housing to rotate relative to the housing about an upper pivot axis 34. An upper rotational element in the form of an upper spool 36 is also supported to rotate about the upper pivot axis 34, with the lower end of the frame 18. Similarly, an upper end of the plate 28 extends inside the housing 32 and is pivotally connected to the housing to rotate relative to the housing about a lower pivot axis 38 and a lower rotational element in the form of a lower spool 40 is supported to rotate about the lower pivot axis, with the upper end of the plate 28. The upper and lower pivot axes 34,38 are generally parallel to each other and extend transverse to the wearer's knee (i.e. extend in a lateral-medial direction).

The upper and lower spools 36,40 are kept a fixed distance apart by the housing 32 and can each rotate relative to the housing, with the upper leg formation 12 and lower leg formation 14, respectively. A flexible tensile element in the form of a steel cable 42 extends partially around the spools 36,40 and crosses between them in a figure-of-eight configuration. The cable 42 is anchored relative to the circumference of each of the spools 36,40 by an anchor element 44, to prevent circumferential slippage of the cable along either spool and as a result, the cable connects the spools for counter rotation relative to the housing. The spools 36,40 and upper and lower leg formations 12,14 are supported for rotation relative to the housing 32 by sealed double bearings—to provide smooth and long lasting pivotal movement.

The double axis pivotal movement in the hinge mechanism 16 replicates human cruciate ligament function and is a much closer simulation to the natural flexion of the wearer's knee, than a simple pivotal movement.

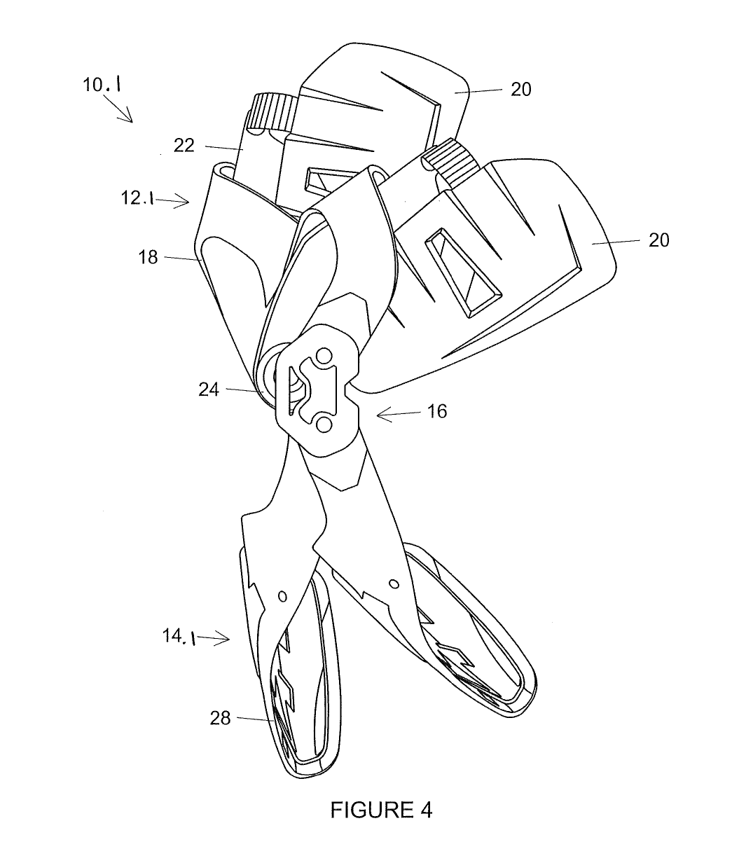

The housing 32 includes stop formations 46 on its anterior side and the stop formations are positioned to contact the front of the lower end of the frame 18 and the front of the upper end of the plate 28, respectively, when the upper leg formation 12 and lower leg formation 14 have pivoted to positions corresponding to maximum permissible knee extension—thus preventing hyperextension (and preventing ACL injury). The stop formations 46 are compressible to provide a soft feel and damping when the knee brace reaches its fully extended position and the positions of the stop formations are adjustable by swapping inserts that form part of the housing 32 (the stop formations 46 forming part of the inserts). The range of pivotal movement that the hinge mechanism 16 allows is shown in FIG. 4, where the upper and lower leg formations 12,14 are shown in flexed and maximum extended positions, relative to the housing 32.

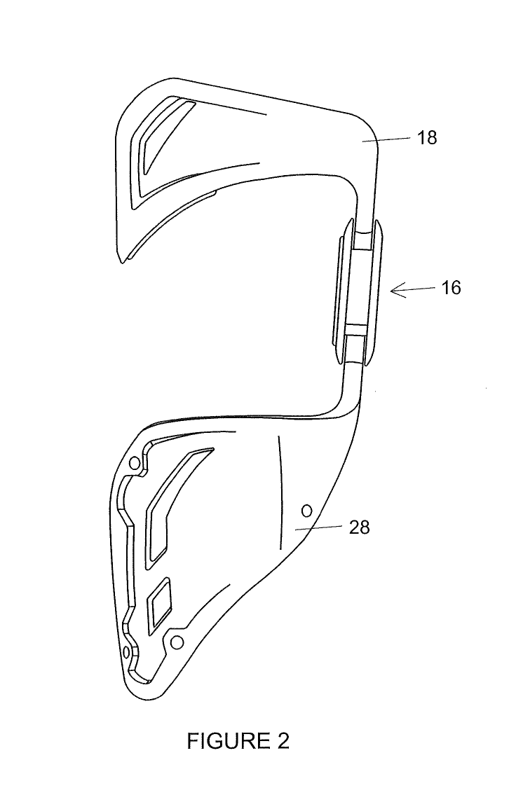

The frame 18, hinge mechanism 16 and plate 28 form a super strong C-shaped structure (shown in FIG. 2), but has no medial hinge mechanism, with the result that the knee brace 10.1 is unlikely to interfere other apparatus during use—especially when compared to prior art knee braces with medial hinges that tended to knock against motorcycles while riding. Further, the use of a single lateral hinge and a knee pad 24 that exerts pressure on the wearer's knee complex at the medial pressure point, allows the tibia to rotate when flexing the knee—more particularly: allows inward rotation of the tibial plateau relative to the femur when extending the knee, and vice versa.

The C-shaped structure shown in FIG. 2 (which includes the plate 28), together with the lateral thigh pad, the lower part of the medial thigh pad 22, and the knee pad 24, form a strong, stiff structure, applying pressure on the three pressure points P1-P3 that stabilise the knee against valgus stress and thus against meniscus and MCL injury. The remainder of formations that can exert pressure on the thigh and calf, do so merely to the extent required to hold the knee brace 10.1 in place—in similar fashion to the attachment of prior art knee pads to wearers' legs.

The knee brace 10.1 preferably includes an integrating sock 48 with a visco-elastic patella cup 50, to provide impact protection and a good fit of the knee brace on the wearer's leg.

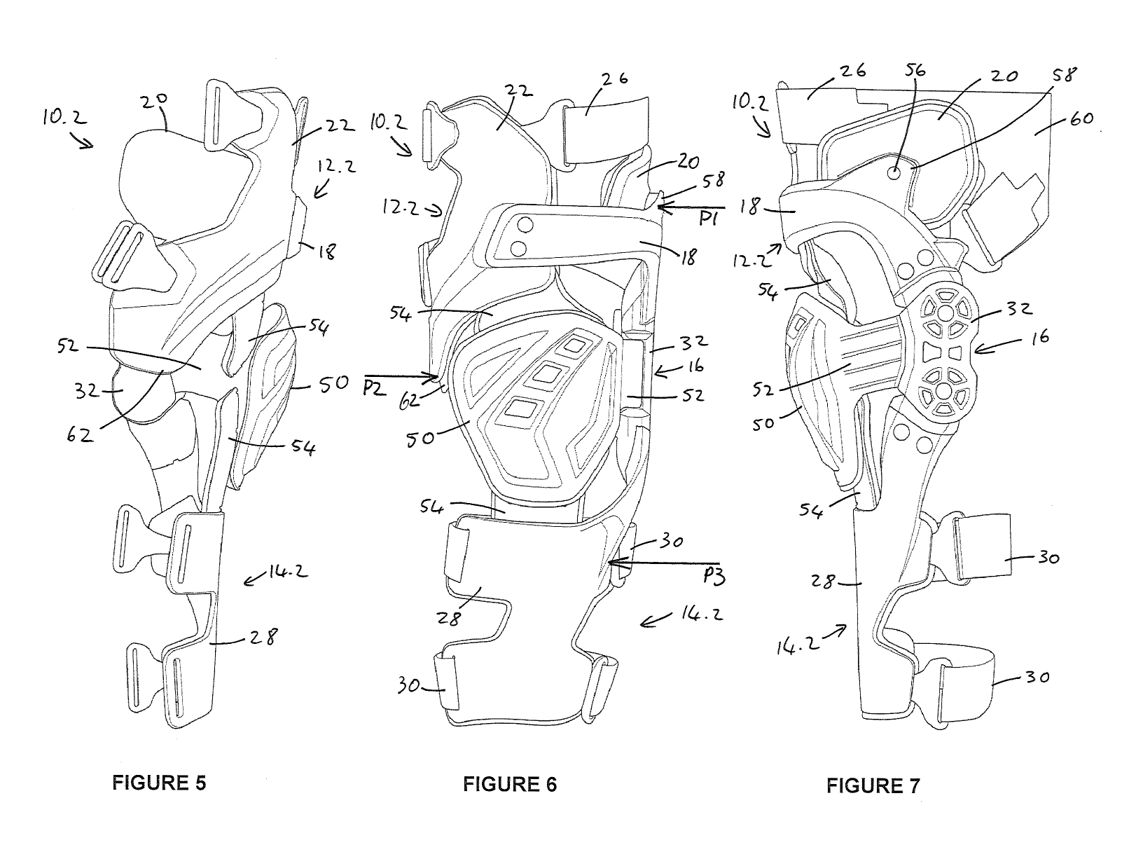

Referring to FIGS. 5 to 7, a second embodiment of a knee brace 10.2 according to the present invention, is substantially similar to the first embodiment shown in

FIGS. 1 to 4, apart from the following differences.

The patella cup 50 is not held in position by a sock, but is held in position by a rigid support arm 52 extending between the housing 32 of the hinge mechanism 16 and the patella cup. Additional protecting plates 54 extend down from the medial thigh pad 22 and up from the plate 28 partly behind the patella cup 50.

The lateral thigh pad 20 is attached to a rigid tab 58 that forms part of the frame 18, by a pivotal pin 56 and this allows the thigh pad 20 to be pivoted and/or rotated about the pin to provide comfort and good load distribution for pressure exerted between the lateral thigh and the frame, at the proximal lateral pressure point P1.

The medial pressure formation of the knee brace 10.2 is not an adjustable knee pad, but instead, the distal end 62 of the medial thigh pad 22 forms a medial pressure formation and presses on the knee complex at the medial pressure point P2.

The plate 28 extends farther posterior in the region of the distal lateral pressure point P3.

The straps 26 holding the upper leg formation in place, are partly substituted and/or supplemented with webbing 60 (shown in FIG. 7) in the form of stretchable fabric that can extend around part of the thigh.

1. A protective device which includes:
an upper leg formation that is attachable to the upper leg of a wearer and that includes an upper lateral pressure formation that presses against the lateral aspect of the wearer's upper leg at a proximal lateral pressure point, when the device is worn;
a lower leg formation that is attachable to the lower leg of the same leg of the wearer and that includes a lower lateral pressure formation that presses against the lateral aspect of the wearer's lower leg, at a distal lateral pressure point, when the device is worn;
a hinge mechanism that is connected to the upper leg formation and to the lower leg formation, said hinge mechanism being configured to allow pivotal movement between the upper leg formation and the lower leg formation about at least one pivot axis-that extends generally transverse to the wearer's knee, said hinge mechanism being disposed entirely on the lateral side of the wearer's knee, when the device is worn; and
a medial pressure formation that forms part of the upper leg formation and that presses against the knee complex at a medial pressure point on the medial aspect of the distal extremity of the femur, when the device is worn;
wherein said upper lateral pressure formation, medial pressure formation and lower lateral pressure formation form a strong, stiff structure which includes the hinge mechanism, said stiff structure applying pressure on the proximal lateral pressure point, the distal lateral pressure point and the medial pressure point that stabilise the knee against valgus stress.

2. The protective device according to claim 1, wherein the hinge mechanism comprises a link that is pivotally connected to the upper leg formation to pivot relative to the upper leg formation about an upper pivot axis, said link also being pivotally connected to the lower leg formation to pivot relative to the lower leg formation about a lower pivot axis, said upper and lower pivot axes extending generally parallel to each other and extending generally transverse to the wearer's knee, when the device is worn.

3. The protective device according to claim 1, wherein the hinge mechanism includes an upper rotational element that is fixed relative to the upper leg formation and a lower rotational element that is fixed relative to the lower leg formation and the upper and lower rotational elements are connected together for counter-rotation.

4. The protective device according to claim 3, wherein the hinge mechanism includes a flexible tensile element that extends partially around the upper and lower rotational elements and crosses between the upper and lower rotational elements, in a figure-of-eight configuration.

5. The protective device according to claim 1, wherein the lower leg formation comprises a thin plate of stiff material.

6. The protective device according to claim 1, wherein the upper leg formation includes elongate tensile elements that extend across the posterior aspect of the wearer's thigh in a crossed configuration, when the device is worn.

7. The protective device according to claim 1, wherein the lower leg formation includes elongate tensile elements that extend across the posterior and lateral aspects of the wearer's calf in a crossed configuration, when the device is worn.
